(19) United States
(12) Patent Application Publication
(10) Pub. No.: US 2008/0319194 A1
(43) Pub. Date: Dec. 25, 2008

(54) Process for Preparing Quinazolinone Derivatives

(76) Inventors: Albrecht Jacobi, Frankfurt (DE); Michael Schul, Plaidt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/993,696

(22) PCT Filed: Jun. 13, 2006
(86) PCT No.: PCT/EP2006/063127

§ 371 Date: Mar. 21, 2008

(30) Foreign Application Priority Data
Jul. 4, 2005 (EP) 05106036.6

(51) Int. Cl. C07D 239/91

(52) U.S. Cl. 544/287

(57) ABSTRACT

The invention relates to a method for producing quinazolinone derivatives of general formula (I), wherein the radicals R¹ to R³ have the meanings indicated in the claims and the description.

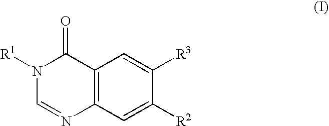

The invention relates to a process for preparing quinazolinone derivatives of general formula (I)

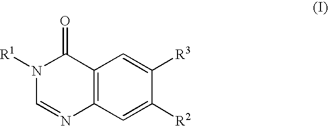

wherein the groups R¹, R² and R³ have the meanings given in the claims and specification.

### BACKGROUND TO THE INVENTION

Quinazolinone derivatives are known from the prior art intermediates for preparing substituted quinazoline derivatives. WO 2004/108664 describes quinazolinone derivatives for preparing quinazoline derivatives, and the use thereof for the treatment of tumoral diseases, diseases of the lungs and airways.

A process for preparing quinazolin-4(3H)-ones using a Yb(OTf)₃ catalyst is described in the literature (Synthesis 2003, 8, 1241).

The aim of the present invention is to provide an improved process for preparing the quinazolinone derivatives according to the invention.

### DETAILED DESCRIPTION OF THE INVENTION

The present invention solves the problem stated above by the method of synthesis described hereinafter, which unlike the method described in WO 2004/108664 and the method known from the literature is a process which is in particular more economical and suitable for large-scale production.

The invention thus relates to a process for preparing compounds of general formula (I),

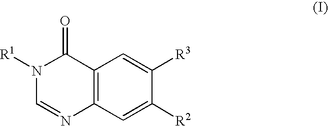

wherein
R¹ denotes a group selected from among benzyl, (R)-(+)-1-phenylmethyl, 4-methoxybenzyl, 4,4′-dimethoxybenzhydryl, 2,4-dimethoxybenzyl, methoxymethyl, benzyloxymethyl, (2-methoxyethyl)oxymethyl, (2-trimethylsilylethyl)oxymethyl and pivaloyloxymethyl, preferably benzyl, (R)-(+)-1-phenylmethyl, 4-methoxybenzyl, 4,4′-dimethoxybenzhydryl- and 2,4-dimethoxybenzyl, particularly preferably benzyl, (R)-(+)-1-phenylmethyl- and 4-methoxybenzyl, particularly preferably benzyl,
   R², R³ independently of one another denote a group selected from among a hydrogen atom,
   a hydroxy group, a benzyl group, a C₁₋₃-alkyloxy group,
   a C₂₋₄-alkyloxy group which is substituted by a group R⁴, where
   R⁴ denotes a hydroxy, C₁₋₃-alkyloxy, C₃₋₆-cycloalkyloxy, di-(C₁₋₃-alkyl)amino, bis-(2-methoxyethyl)-amino, pyrrolidin-1-yl, piperidin-1-yl, homopiperidin-1-yl, morpholin-4-yl, homomorpholin-4-yl, 2-oxa-5-aza-bicyclo[2.2.1]hept-5-yl, 3-oxa-8-aza-bicyclo[3.2.1]oct-8-yl, 8-oxa-3-aza-bicyclo[3.2.1]oct-3-yl, 4-C₁₋₃-alkyl-piperazin-1-yl or 4-C₁₋₃-alkyl-homopiperazin-1-yl group, while the above-mentioned pyrrolidinyl, piperidinyl, piperazinyl and morpholinyl groups may each be substituted by one or two C₁₋₃-alkyl groups, particularly preferably a hydroxy group or a C₁₋₃-alkyloxy group, particularly preferably a hydroxy group or a methoxy group,
      a C₃₋₇-cycloalkyloxy or C₃₋₇-cycloalkyl-C₁₋₃-alkyloxy group,
      a tetrahydrofuran-3-yloxy, tetrahydropyran-3-yloxy or tetrahydropyran-4-yloxy group, and
      a tetrahydrofuranyl-C₁₋₃-alkyloxy or tetrahydropyranyl-C₁₋₃-alkyloxy group,
      optionally in the form of the tautomers, the racemates, the enantiomers, the diastereomers and the mixtures thereof, and optionally the pharmacologically acceptable acid addition salts thereof,
      characterised in that
      (a) a compound of formula (IV)

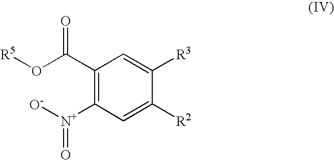

wherein R² and R³ are as hereinbefore defined,
and R⁵ denotes a group selected from among C₁-C₅-alkyl, benzyl, benzhydryl, p-nitrobenzyl and allyl, preferably methyl or ethyl, particularly preferably methyl, is hydrogenated with hydrogen in the presence of a hydrogenation catalyst, and
(b) the compound of general formula (II) resulting from step (a)

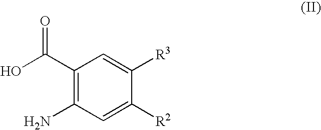

wherein R² and R³ have the meanings specified
is reacted with a compound of general formula (III)

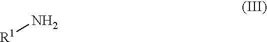

wherein R¹ is as hereinbefore defined,
and triethyl orthoformate or trimethyl orthoformate, particularly preferably triethyl orthoformate.

The invention further relates to a process for preparing compounds of general formula (I),

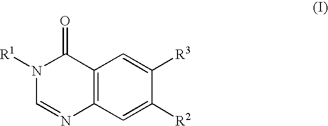

wherein R¹ to R³ may have the above specified meanings,
optionally in the form of the tautomers, the racemates, the enantiomers, the diastereomers and the mixtures thereof, and optionally the pharmacologically acceptable acid addition salts thereof,
characterised in that a compound of general formula (II)

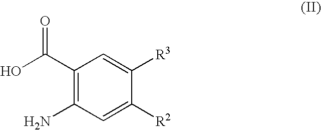

wherein R² and R³ may have the above specified meanings,
is reacted with a compound of general formula (III)

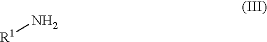

wherein R¹ may have the above specified meanings,
and triethyl orthoformate or trimethyl orthoformate, preferably triethyl orthoformate. The compound of formula (III) and the orthoformate may be added to the reaction mixture simultaneously or successively. Preferably the compound of formula (III) is added to the reaction mixture first, followed by the orthoformate.

The invention further relates to a process for preparing of general formula (II), wherein R² and R³ may have the above specified meanings,

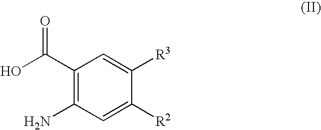

characterised in that a compound of formula (IV)

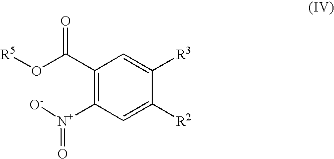

wherein R² and R³ may have the above specified meanings,
and R⁵ denotes a group selected from among C₁-C₅-alkyl, benzyl, benzhydryl, p-nitrobenzyl and allyl, preferably methyl or ethyl, particularly preferably methyl,
is hydrogenated with hydrogen in the presence of a hydrogenation catalyst.

A process in which Pd/C or Raney nickel, preferably Pd/C, is used as the hydrogenation catalyst is preferred.

Also preferred is a process wherein the amount of added hydrogenation catalyst is within in the range from 0.1 to 10 wt.-%, preferably from 1 to 5 wt.-%, particularly preferably from 2 to 3 wt.-%, based on the compound of formula (IV) used.

Also preferred is a process in which the reaction temperature is in the range from 20° C. to 60° C., preferably from 30 to 55° C., particularly preferably from 45 to 50° C.

Also preferred is a process in which the hydrogen pressure is 1 bar to 100 bar, preferably 2 to 50 bar, particularly preferably 3 to 5 bar.

Particularly preferred is a process wherein

R¹ denotes benzyl.

Particularly preferred is a process wherein

R², R³ independently of one another represent OH or OMe.

The invention further relates to compounds of general formula (I),

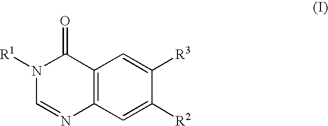

wherein
R¹-R³ may have the above specified meanings,
where
R³ may not represent OH if
R¹ denotes a group selected from among benzyl, 2,4-dimethoxybenzyl, methoxymethyl, benzyloxymethyl, (2-methoxyethyl)oxymethyl, (2-trimethylsilylethyl)oxymethyl and pivaloyloxymethyl,
optionally in the form of the tautomers, the racemates, the enantiomers, the diastereomers and the mixtures thereof, and optionally the pharmacologically acceptable acid addition salts thereof.

The invention further relates to compounds according to general formula (II),

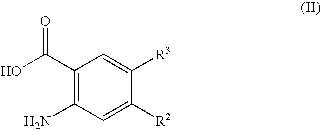

wherein R² and R³ may be as hereinbefore defined.

Suitable solvents for the reaction are solvents such as e.g. water, amides such as dimethylformamide, dimethylacetamide, N-methylpyrrolidinone or sulphoxides such as e.g. dimethylsulphoxide, sulpholane or primary alcohols such as e.g. ethanol, 1-propanol, 1-butanol, 1-pentanol or secondary alcohols such as e.g. 2-propanol, 2-butanol or the isomeric secondary alcohols of pentane or hexane or tertiary alcohols such as e.g. Tert-butanol or nitriles such as e.g. Acetonitrile or 2-propylnitrile. It is particularly preferable to carry out the reaction in water.

The reactions are worked up by the usual methods, e.g. By extractive purification steps or precipitation and crystallisation procedures.

The compounds according to the invention may be present in the form of the individual optical isomers, mixtures of the individual enantiomers, diastereomers or racemates, in the form of the tautomers and in the form of the free bases or the corresponding acid addition salts with pharmacologically acceptable acids—such as for example acid addition salts with hydrohalic acids, for example hydrochloric or hydrobromic acid, or organic acids, such as for example oxalic, fumaric, diglycolic or methanesulphonic acid.

By alkyl groups and alkyl groups, which are part of other groups, are meant branched and unbranched alkyl groups with 1 to 3 carbon atoms, preferably 1 to 2 carbon atoms, particularly preferably 1 carbon atom; examples include methyl, ethyl, n-propyl and isopropyl.

In the above-mentioned alkyl groups one or more hydrogen atoms may optionally be replaced by other groups. For example, these alkyl groups may be substituted by the halogen atoms fluorine, chlorine, bromine or iodine. The substituents fluorine and chlorine are preferred. The substituent chlorine is particularly preferred. All the hydrogen atoms of the alkyl group may optionally be replaced. Examples of cycloalkyl groups include saturated or unsaturated cycloalkyl groups with 3 to 7 carbon atoms, for example cyclopropyl, cyclobutyl, cyclopentyl, cyclopentenyl, cyclohexyl, cyclohexenyl or cycloheptyl, preferably cyclopropyl, cyclopentyl or cyclohexyl, while each of the above-mentioned cycloalkyl groups may optionally also carry one or more substituents.

The substituent R¹ may represent a group selected from among benzyl, (R)-(+)-1-phenylmethyl, 4-methoxybenzyl, 4,4′-dimethoxybenzhydryl, 2,4-dimethoxybenzyl, methoxymethyl, benzyloxymethyl, (2-methoxyethyl)oxymethyl, (2-trimethylsilylethyl)oxymethyl and pivaloyloxymethyl, preferably benzyl, (R)-(+)-1-phenylmethyl, 4-methoxybenzyl, 4,4′-dimethoxybenzhydryl or 2,4-dimethoxybenzyl, particularly preferably benzyl, (R)-(+)-1-phenylmethyl, 4-methoxybenzyl, particularly preferably benzyl.

The substituent R² may denote a group selected from among a hydrogen atom, a hydroxy group, a C₁₋₃-alkyloxy group, a C₂₋₄-alkyloxy group which is substituted by a group R⁴, where
R⁴ denotes a hydroxy, C₁₋₃-alkyloxy, C₃₋₆-cycloalkyloxy, di-(C₁₋₃-alkyl)amino, bis-(2-methoxyethyl)-amino, pyrrolidin-1-yl, piperidin-1-yl, homopiperidin-1-yl, morpholin-4-yl, homomorpholin-4-yl, 2-oxa-5-aza-bicyclo[2.2.1]hept-5-yl, 3-oxa-8-aza-bicyclo[3.2.1]oct-8-yl, 8-oxa-3-aza-bicyclo[3.2.1]oct-3-yl, 4-C₁₋₃-alkyl-piperazin-1-yl or 4-C₁₋₃-alkyl-homopiperazin-1-yl group, while the above-mentioned pyrrolidinyl, piperidinyl, piperazinyl and morpholinyl groups may each be substituted by one or two C₁₋₃-alkyl groups,
   a C₃₋₇-cycloalkyloxy or C₃₋₇-cycloalkyl-C₁₋₃-alkyloxy group,
   a tetrahydrofuran-3-yloxy, tetrahydropyran-3-yloxy or tetrahydropyran-4-yloxy group, and
   a tetrahydrofuranyl-C₁₋₃-alkyloxy or tetrahydropyranyl-C₁₋₃-alkyloxy group, particularly preferably a hydroxy group or a C₁₋₃-alkyloxy group, particularly preferably a hydroxy group or a methoxy group, most preferably a methoxy group.

The substituent R³ may denote a group selected from among a hydrogen atom, a hydroxy group, a C₁₋₃-alkyloxy group, a C₂₋₄-alkyloxy group which is substituted by a group R⁴, where
R⁴ denotes a hydroxy, C₁₋₃-alkyloxy, C₃₋₆-cycloalkyloxy, di-(C₁₋₃-alkyl)amino, bis-(2-methoxyethyl)-amino, pyrrolidin-1-yl, piperidin-1-yl, homopiperidin-1-yl, morpholin-4-yl, homomorpholin-4-yl, 2-oxa-5-aza-bicyclo[2.2.1]hept-5-yl, 3-oxa-8-aza-bicyclo[3.2.1]oct-8-yl, 8-oxa-3-aza-bicyclo[3.2.1]oct-3-yl, 4-C₁₋₃-alkyl-piperazin-1-yl or 4-C₁₋₃-alkyl-homopiperazin-1-yl group, while the above-mentioned pyrrolidinyl, piperidinyl, piperazinyl and morpholinyl groups may each be substituted by one or two C₁₋₃-alkyl groups,
   a C₃₋₇-cycloalkyloxy or C₃₋₇-cycloalkyl-C₁₋₃-alkyloxy group,
   a tetrahydrofuran-3-yloxy, tetrahydropyran-3-yloxy or tetrahydropyran-4-yloxy group, and
   a tetrahydrofuranyl-C₁₋₃-alkyloxy or tetrahydropyranyl-C₁₋₃-alkyloxy group, particularly preferably a hydroxy group or a C₁₋₃-alkyloxy group, particularly preferably a hydroxy group or a methoxy group, most preferably a hydroxy group.

The compound of formula (IV) is commercially available and may be obtained e.g. from Sigma-Aldrich. It may be prepared by methods known from the literature (P. Carpenter et al., J. Chem. Soc. Perkin Trans. 1 (1979), 103).

The compounds according to the invention may be prepared using the synthesis methods described below, while the substituents of general formulae (I) to (IV) may have the above-mentioned meanings. These methods are intended as an illustration of the invention without restricting it to their content.

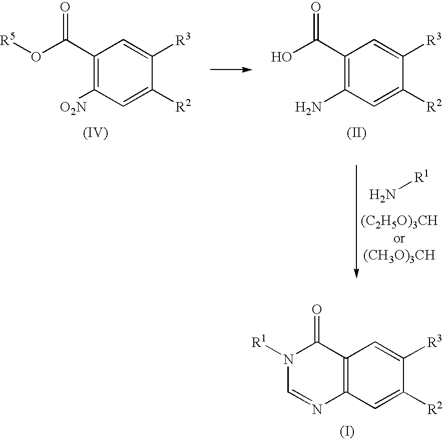

A compound of formula (IV) is hydrogenated to form the compound of formula (II) (Step 1).

Then the compound of formula (II) is reacted to form the compound of formula (I) (Step 2). The compound (1V) is commercially obtainable (e.g. from Sigma-Aldrich).

In Step 1, 2 to 5 equivalents, preferably 3.5 equivalents of a base, preferably potassium hydroxide, sodium hydroxide, particularly preferably potassium hydroxide, are stirred in a diluent, for example water, ethanol, preferably water. 1 equivalent of compound (1V) is added to this mixture and the reaction mixture is refluxed with stirring. The reaction mixture is refluxed for another 3 to 5 hours, preferably 4 hours, with stirring, while methanol is eliminated by distillation. Then the pH is adjusted to 8.5 to 10, preferably pH 9, with acetic acid. The resulting mixture is hydrogenated with hydrogen in the presence of a hydrogenation catalyst, for example Pd/C, Raney nickel, preferably Pd/C, in an amount of 0.1 to 10 wt.-% based on the compound (1V) put in, preferably 1 to 5 wt.-%, particularly preferably 2-3 wt.-%, at a temperature of 20° C. to 60° C., preferably 45° C. to 55° C., particularly preferably 50° C., and at a hydrogen pressure of 1 bar to 100 bar, preferably 2 to 50 bar, particularly preferably 3 to 5 bar, until the hydrogen uptake stops. Acetic acid is added to the resulting hydrogenated solution under protective gas until a pH of 4 to 7, preferably pH 6 is achieved. During this procedure the compound (II) is precipitated out. It is isolated and then dried in vacuo for 6 to 18 hours, preferably 12 hours, at 30° C. to 70° C., preferably 50° C.

The compound (II) may be used in Step 2 without any preliminary purification. In Step 2, 1 equivalent of compound (II) is suspended under protective gas in an organic solvent, for example ethanol, isopropanol, toluene, dioxane, acetonitrile, N-methyl-2-pyrrolidinone, triethyl orthoformate, trimethyl orthoformate, preferably ethanol, and refluxed with stirring. 1 to 1.5 equivalents, preferably 1.05 equivalents of an amine, for example benzylamine, (R)-(+)-1-phenylmethylamine, 4-methoxybenzylamine, 2,4-dimethoxybenzylamine, 4,4′-dimethoxybenzhydrylamine, preferably benzylamine, are metered in at reflux temperature. Then 2 to 10 equivalents, preferably 2.4 to 3 equivalents of a trialkyl orthoformate, for example triethyl orthoformate, trimethyl orthoformate, preferably triethyl orthoformate, are added while refluxing. The resulting reaction mixture is stirred for another 2 to 10 hours, preferably 4 hours while refluxing. Then the temperature of the reaction mixture is adjusted to 10° C. to 40° C., preferably 20° C. and the mixture is stirred for another 10 to 120 minutes, preferably 30 minutes at this temperature. The suspension is isolated and compound (1) thus obtained is dried in vacuo for 6 to 18 hours, preferably 12 hours at 30° C. to 70° C., preferably 50° C.

The compounds of general formula (I) be synthesised analogously to the synthesis examples that follow. These Examples are, however, intended only as an exemplifying procedure to illustrate the invention further without restricting it to the content thereof.

### EXAMPLE 1

### Synthesis of 3-benzyl-3,4-dihydro-4-oxo-6,7-dimethoxy-quinazoline (3)

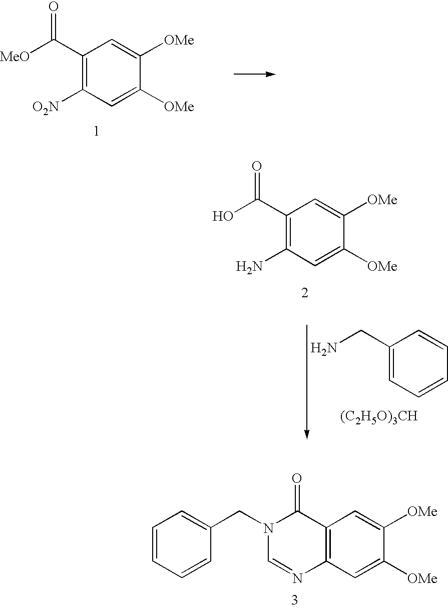

The compound I is commercially available and may be obtained for example from Sigma-Aldrich (CAS-No. 26791-93-5).

### Step A:

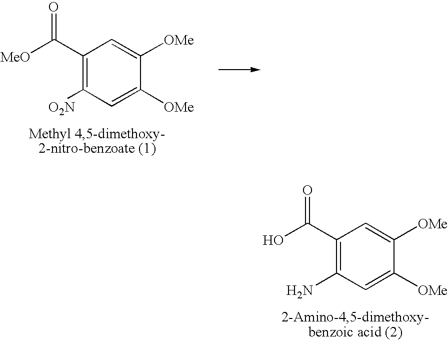

48.13 g (0.729 mol) of KOH pellets (w=85%) are dissolved in 250 ml of ice water. 50 g (0.207 mol) methyl-4,5-dimethoxy-2-nitro-benzoate (1) are added to the clear solution and the resulting green suspension is heated to 70° C. During the heating a dark red solution is formed. Once the reaction has ended (monitored by HPLC) the solution is cooled to ambient temperature and adjusted to pH 6.6 with 34.6 g (0.570 mol) glacial acetic acid. The resulting red suspension is hydrogenated with 1 g of 10% Pd/C at 50° C. and 3.5 bar until the reaction comes to a standstill. Then the hydrogenation solution is filtered off and adjusted to pH 5.1 with 31.82 g (0.525 mol) glacial acetic acid under an inert gas. The light green suspension is stirred for 30 min at RT, then cooled to 5° C. and stirred for another 30 min.

The product (2) is filtered off, washed in two batches with a total of 250 ml of ice water and then dried at 55° C. for 12 h in a vacuum drying cupboard.

This reaction yielded 35.18 g (0.173 mol, 83% of theory) of light grey crystals.

### Step B:

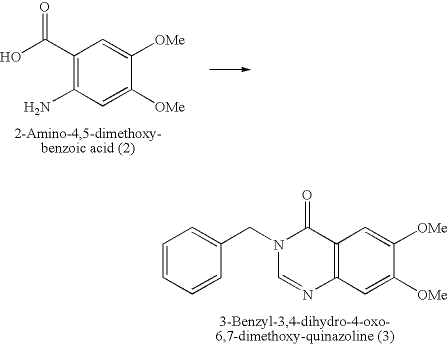

20 g (0.101 mol) of compound (2) is suspended under an inert gas in 125 ml of ethanol and refluxed. 11.41 g (0.106 mol) benzylamine are metered in while refluxing. Then 36.08 g (0.243 mol) triethyl orthoformate is metered in. The resulting brown suspension is stirred for 3.5 h at 80° C. After the conversion is complete (monitored by HPLC) the suspension is cooled to RT and stirred for 30 min. The product (3) is filtered off and washed with 25 ml of ethanol in two batches. The crystalline product is dried for 12 h in the vacuum dryer at 55° C. The reaction yielded 26.51 g (0.088 mol, 88% of theory) of colourless crystals.

### EXAMPLE 2

### Synthesis of 3-benzyl-3,4-dihydro-4-oxo-6-hydroxy-7-methoxy-quinazoline (3)

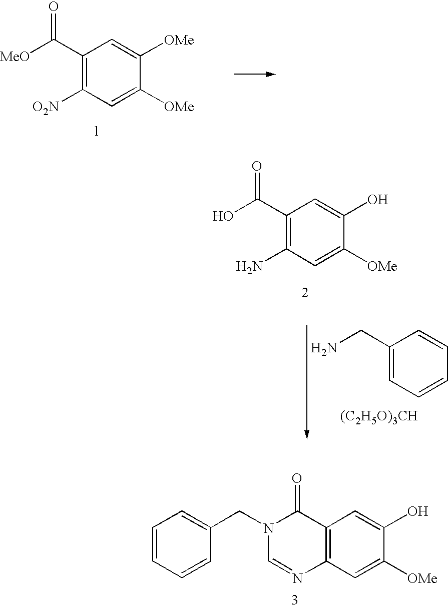

The compound 1 is commercially available and may be obtained for example from Sigma-Aldrich (CAS-No. 26791-93-5).

### Step A:

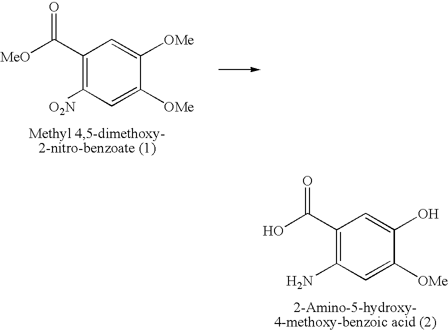

770 g (11.665 mol) of KOH pellets (w=85%) are dissolved in 4000 ml of ice water. 800 g (3.317 mol) methyl-4,5-dimethoxy-2-nitro-benzoate (1) are added to the clear solution and the resulting green suspension is refluxed. During the heating a red solution is formed. The solution is refluxed with stirring for about 4 h while distilling off 850 ml of methanol/water. Once the reaction is complete (monitored by HPLC) the solution is cooled to ambient temperature and adjusted to pH 9 with 337.6 g (5.566 mol) glacial acetic acid. The nitro group reduction and isolation of the product (2) were carried out analogously to Ex. 1.

The reaction yielded 558.5 g (3.049 mol, 92% of theory) in the form of grey crystals.

### Step B:

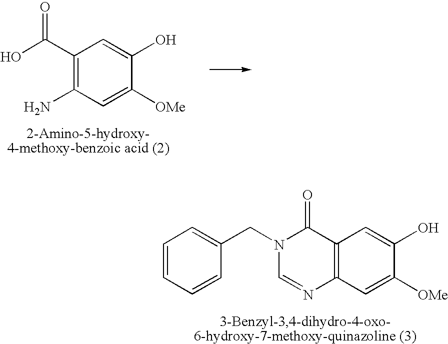

The reaction of 536.4 g (2.929 mol) of compound (2) was carried out analogously to Step B in Ex. 1. The reaction yielded 752.3 g (91% of theory) in the form of beige crystals.

### EXAMPLE 3

### Synthesis of 3-(4-methoxy-benzyl)-3,4-dihydro-4-oxo-6-hydroxy-7-methoxy-quinazoline (3)

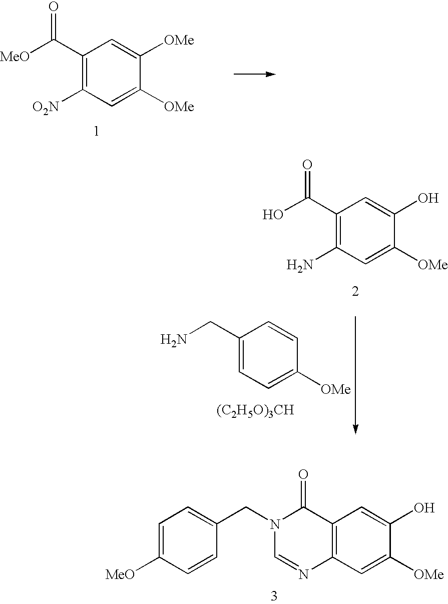

The compound I is commercially available and may be obtained for example from Sigma-Aldrich (CAS-No. 26791-93-5).

Step A was carried out analogously to Step A in Ex. 1.

### Step B:

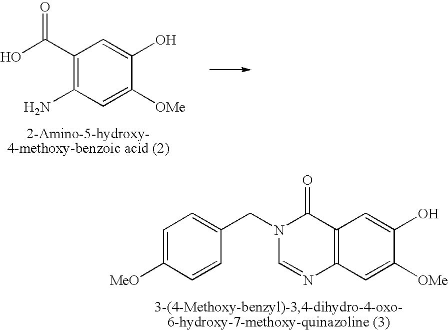

1 g (0.005 mol) of compound (2) is suspended in 10 ml of ethanol under inert gas and refluxed. 0.79 g (0.006 mol) 4-methoxy-benzylamine is metered in while refluxing. Then 1.94 g (0.013 mol) triethyl orthoformate is metered in. The resulting grey suspension is stirred for 3.5 h at 80° C. The suspension is cooled to RT and stirred for 30 min. The product (3) is filtered off and washed with 5 ml of ethanol. The crystalline product is dried for 12 h in the vacuum dryer at 55° C. The reaction yielded 1.28 g (0.004 mol, 74.9% of theoretical) of beige crystals.

The compounds of formula (I) listed in Table 1, inter alia, were obtained analogously to the method described above.

TABLE 1

| | |
|---|---|
| | (I) |
| 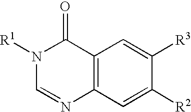 | |

| Example | R¹ | R² | R³ |
|---|---|---|---|
| 4 | 1-(R)-phenyl- | methoxy | hydroxy |
| | methyl- | | |
| 5 | 4,4′- | methoxy | hydroxy |
| | dimethoxy- | | |
| | benzhydryl | | |
| 6 | phenyl-methyl- | methoxy | 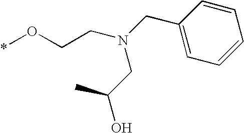 |
| 7 | phenyl-methyl- | methoxy | 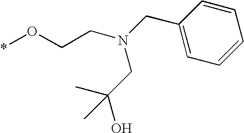 |

1. Process for preparing compounds of general formula (I), 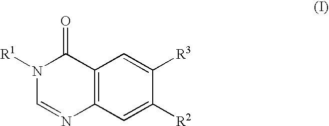
wherein
R¹ denotes a group selected from among benzyl, (R)-(+)-1-phenylmethyl, 4-methoxybenzyl, 4,4′-dimethoxybenzhydryl, 2,4-dimethoxybenzyl, methoxymethyl, benzyloxymethyl, (2-methoxyethyl)oxymethyl, (2-trimethylsilylethyl)oxymethyl and pivaloyloxymethyl,
R², R³ independently of one another denote a group selected from among a hydrogen atom, a hydroxy group, a benzyl group, a C₁₋₃-alkyloxy group,
a C₂₋₄-alkyloxy group which is substituted by a group R⁴, where
R⁴ denotes a group selected from among hydroxy, C₁₋₃-alkyloxy, C₃₋₆-cycloalkyloxy, di-(C₁₋₃-alkyl)amino, bis-(2-methoxyethyl)-amino, pyrrolidin-1-yl, piperidin-1-yl, homopiperidin-1-yl, morpholin-4-yl, homomorpholin-4-yl, 2-oxa-5-aza-bicyclo[2.2.1]hept-5-yl, 3-oxa-8-aza-bicyclo[3.2.1]oct-8-yl, 8-oxa-3-aza-bicyclo-[3.2.1]oct-3-yl, 4-C₁₋₃-alkyl-piperazin-1-yl and 4-C₁₋₃-alkyl-homopiperazin-1-yl group, while the above-mentioned pyrrolidinyl, piperidinyl, piperazinyl and morpholinyl groups may each be substituted by one or two C₁₋₃-alkyl groups,
a C₃₋₇-cycloalkyloxy or C₃₋₇-cycloalkyl-C₁₋₃-alkyloxy group,
a tetrahydrofuran-3-yloxy, tetrahydropyran-3-yloxy or tetrahydropyran-4-yloxy group, and
a tetrahydrofuranyl-C₁₋₃-alkyloxy or tetrahydropyranyl-C₁₋₃-alkyloxy group,
optionally in the form of the tautomers, the racemates, the enantiomers, the diastereomers and the mixtures thereof, and optionally the pharmacologically acceptable acid addition salts thereof,
characterised in that
(a) a compound of formula (IV) 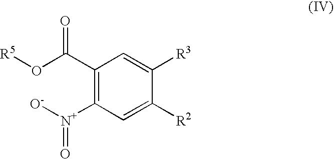
wherein R² and R³ have the meanings specified,
and R⁵ denotes a group selected from among C₁-C₅-alkyl, benzyl, benzhydryl, p-nitrobenzyl and allyl,
is hydrogenated with hydrogen in the presence of a hydrogenation catalyst, and
(b) the compound of general formula (II) resulting from step (a) 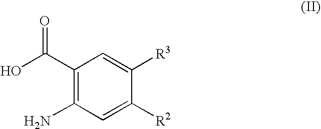
wherein R² and R³ have the meanings specified
is reacted with a compound of general formula (III) 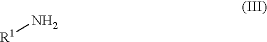
wherein R¹ is as hereinbefore defined,
and triethyl orthoformate or trimethyl orthoformate.

2. Process for preparing compounds of general formula (I), 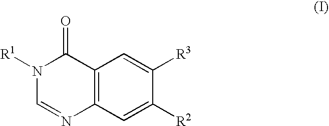
wherein R¹ to R³ may have the above meanings,
optionally in the form of the tautomers, the racemates, the enantiomers, the diastereomers and the mixtures thereof, and optionally the pharmacologically acceptable acid addition salts thereof,
characterised in that a compound of general formula (II) 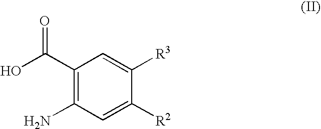
wherein R² and R³ may have the above meanings,
is reacted with a compound of general formula (III) 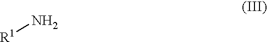
wherein R¹ may have the above meanings,
and triethyl orthoformate or trimethyl orthoformate.

3. Process for preparing compounds of general formula (II),
wherein R² and R³ may have the meanings specified, 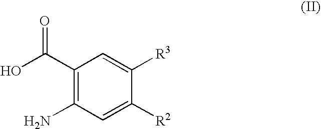
characterised in that a compound of formula (IV) 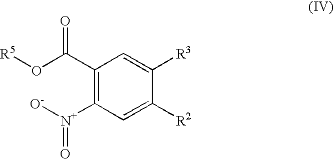
wherein R², R³ and R⁵ may have the meanings specified,
is hydrogenated with hydrogen in the presence of a hydrogenation catalyst.

4. Process according to claim 1, wherein Pd/C or Raney nickel is used as hydrogenation catalyst.

5. Process according to claim 1, characterised in that the amount of added hydrogenation catalyst is in the range from 0.1 to 10 wt.-%, based on the compound of formula (IV) used.

6. Process according to claim 2, characterised in that the reaction temperature is in the range from 20° C. to 60° C.

7. Process according to claim 3, characterised in that the hydrogen pressure is from 1 bar to 100 bar.

8. Process according to claim 1, wherein
R¹ denotes benzyl.

9. Process according to claim 1, wherein
R², R³ independently of one another represent OH or OMe.

10. Compounds according to general formula (I), 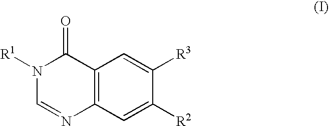
wherein
R¹-R³ may have the meanings specified,
wherein
R³ may not represent OH if
R¹ denotes a group selected from among benzyl, 2,4-dimethoxybenzyl, methoxymethyl, benzyloxymethyl, (2-methoxyethyl)oxymethyl, (2-trimethylsilylethyl)oxymethyl and pivaloyloxymethyl,
optionally in the form of the tautomers, the racemates, the enantiomers, the diastereomers and the mixtures thereof, and optionally the pharmacologically acceptable acid addition salts thereof.

11. Compounds according to general formula (II), 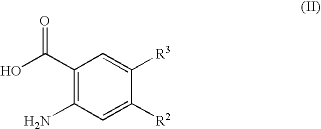
wherein R¹ to R³ may have the meanings specified.
